(19) United States
(12) Patent Application Publication
(10) Pub. No.: US 2005/0026860 A1
(43) Pub. Date: Feb. 3, 2005

(54) Nucleolin antisense sequence for inhibition of cancer cell proliferation

(76) Inventors: Chin-Tarng Lin, Da-an District Taipei City (TW); Han-Chung Wu, Songshan District Taipei City (TW)

Correspondence Address: OSTROLENK FABER GERB & SOFFEN, 1180 AVENUE OF THE AMERICAS, NEW YORK, NY, 100368403, US

(21) Appl. No.: 10/797,822

(22) Filed: Mar. 9, 2004

(30) Foreign Application Priority Data
Jul. 30, 2003 (TW) 092120792

(51) Int. Cl. A61K 48/00; C12Q 1/68; C07H 21/02

(52) U.S. Cl. 514/44; 536/23.1

(57) ABSTRACT

Disclosed is a nucleolin antisense oligonucleotide for inhibition of tumor cell proliferation. The nucleolin antisense oligonucleotide comprising 20 nucleotides is complementary to the nucleolin mRNA. When the nucleolin antisense oligonucleotide hybridizes to nucleolin mRNA, it is effective in suppression of nucleolin RNA synthesis and the corresponding protein expression, which subsequently arrests the growth rate of tumor cells. The nucleolin antisense oligonucleotide is effective in inhibition of nucleolin expression of tumor cells in vitro and in vivo. Therefore, the nucleolin antisense oligonucleotide can be used as a therapeutic agent for inhibition of tumor cell proliferation.

### BACKGROUND OF THE INVENTION

1. Field of the Invention

The present invention is related to an oligonucleotide sequence for inhibition of cancer cell proliferation, and specifically to a nucleolin antisense sequence that inhibits cancer cell proliferation. The nucleolin antisense sequence can bind to the nucleolin nucleotide during synthesis of nucleolin, which then achieves the goal of inhibition of tumor cell proliferation.

2. Prior Arts

Nasopharyngeal carcinoma (NPC) is one of the common cancers among Chinese living in South China, Taiwan and Singapore. Until now the etiological factors of the malignant epithelial cancer are still not clearly defined. However, it is generally accepted that nasopharyngeal carcinoma is closely associated with Epstein-Barr virus (EB virus) infection.

It is well known that when EBV infects a host cell, EBV may appear two episomal forms, namely latent infection and lytic infection. In the former one, the virus genome replicates synchronously with the host cell. In the later one, the virus replicates in a rolling circle form, and mass amounts of virions are synthesized to lead to cell lysis.

Most of the infected cells, including B lymphocytes and NPC cells, present in a latent status, with few infected B cells enter lytic status upon induction by interleukin 6 (IL-6) or phorbol ester. The EB virus can be categorized into three types according to its latent gene expression: latency I, in which only Epstein-Barr nuclear antigen 1 (EBNA-1) is expressed without other latent virus protein being expressed and Group I Burkitt's lymphoma (Group I BL) belongs to this type; latency II, in which type I EBNA and latent membrane protein I (LMP 1) virus protein are expressed and NPC, Hodgkin's disease and T cell lymphoma belong to this type; and latency III, in which all of the nine latent virus proteins are expressed and examples are Group III BL and Lymphoblastoid cell line (LCL) cells.

Studies have shown that NPC cells infected with EB virus grow faster than those without infection. Furthermore, the infected tumor cells grow into larger tumor lumps in immunity deficient mouse. The results indicate that EB virus might enhance the propagation ability of the NPC cells. Using the microarray technique to analyze effects of EB virus on change of NPC cell gene profile, shows that nucleolin in the infected cells is expressed at higher level than uninfected cells.

NPC is a human epithelial squamous cancer. At present, major treatments of NPC include radiotherapy, surgical removal, and chemotherapy. Although these treatments are efficacious to patients diagnosed at the initial stage of the disease, there is still about 20 percent of the patients showing recurrence within five years after treatment. For those patients who are diagnosed at the late stage of the disease, chance of recovery after treatment is less than 50%. In addition, side effects have been observed in association with the treatments described above and high recovery rate is also limited. Therefore, it is important to develop a more effective adjuvant therapy that improves the survival rate of late stage patients and minimizes the side effects and pain.

Application of the gene therapy to treat human disease has entered clinical practice. Scientists have devoted to researches of the gene therapy, expecting that the gene therapy will become a novel therapeutic technique, which may solve problems encountered today, such as non-specificity, death of normal cells, and side effects.

Nucleolin, first described by Orrick et al (1973), is a protein with molecular weight about 100-110 kDa, and mainly existing in the nucleus of the propagating cells. Nucleolin exhibits auto-degradation and shows two degraded bands about 70 and 50 kDa in Western blotting analysis. Nucleolin is highly phosphorylated and methylated, and can be ADP-ribosylated. Because synthesis of the nucleolin is positively correlated with increased rate of cell division, tumor cells and rapidly dividing cells have higher levels of nucleolin content.

It is believed that expression level of nucleolin correlates with cell proliferation rate. Nucleolin levels are highest in tumors and moderate in other rapidly dividing cells. It can be used in studies of different cancer cell lines as useful marker for cell proliferation. Since nucleolin plays a vital role in tumor cell proliferation, the present invention provides a strategy of inhibition of nucleolin expression to suppress the growth rate of tumor cells.

### SUMMARY OF THE INVENTION

The present invention provides an antisense nucleotide which binds to the nucleolin nucleotide and subsequently inhibits proliferation of tumor cells.

Reports show that nucleolin levels are high in tumor cells and rapidly dividing cells, and EB virus enhances the synthesis of nucleolin. To confirm that nucleolin expression is up-regulated in NPC cells, Western blot is employed to analyze expression of nucleolin protein in tumor cell lines. The results show that tumor cell lines display strong staining band of nucleolin and two degraded bands, whereas the normal epithelial cells show weak, if any of the nucleolin. Therefore, it is confirmed that nucleolin indeed is positively correlated to the cell proliferation, and inhibition of nucleolin synthesis provides opportunity for suppression of tumor cell proliferation.

To prove the assumption that inhibition of nucleolin synthesis provides opportunity for suppression of tumor cell proliferation as stated above, the present invention, based on the nucleolin mRNA sequence, designs and synthesizes a complementary nucleolin antisense sequence, the phosphorothioate oligonucleotide (in short, S-oligos). It is expected that, through specific binding of the nucleolin antisense S-oligos to nucleolin mRNA, translation process shall be prohibited, which consequently leads to inhibition of the nucleolin expression.

Experiments show that the nucleolin antisense S-oligos is effective on treatment of tumor cells. It is also found that only a small amount of the antisense S-oligos is needed to inhibit tumor cell growth. Furthermore, the nucleolin antisense S-oligos treated tumor cells have very low levels of nucleolin mRNA and nucleolin protein expression.

Tumor cells treated with the S-oligos also show time-dependent apoptotic changes. Most of the tumor cells appear apoptosis after 2-3 days, 86% of the tumor cells are killed after 4 days of treatment. While the controlled group (which is treated with the sense strand S-oligos) shows no sign of effect on cell growth, the results strongly support the suggestion that inhibition of nucleolin expression may be the best approach to inhibit tumor cell growth in vitro.

In vivo, inhibition of nucleolin expression by the nucleolin antisense S-oligos is also examined in mouse model implanted with NPC cells. The results prove the growth of tumor cells are also prohibited significantly.

Based on the description above, the present invention clearly demonstrates. that the nucleolin antisense nucleotide is effective on inhibition of nucleolin synthesis and its protein expression, in which tumor cell proliferation is prohibited consequently, and thus providing an effective method for NPC treatment.

The present invention will be further explained in the following examples, These examples should not, however, be considered to limit the scope of the invention, which is defined by the appended claims. Those skilled in the art may modify the present invention upon review of this specification within the scope of the present invention.

### BRIEF DESCRIPTION OF THE DRAWINGS

FIG. 1(A) shows Western blot analysis of nucleolin expression in normal mucosal epithelial cells and NPC cells.

FIG. 1(B) shows Western blotting of nucleolin expression in normal mucosal cells and non-NPC cells.

FIG. 1(C) shows immunohistochemical localization of nucleolin in NPC biopsy specimen.

FIG. 2 demonstrates the nucleolin antisense/sense sequence and effect of nucleolin antisense on the expression of nucleolin in NPC-TW01 cells.

FIG. 2(A) shows the S-oligos design: nucleolin antisense/sense phosphorothioate oligonucleotide.

FIG. 2(B) shows expression of nucleolin in S-oligos treated NPC cells, which is analyzed by RT-PCR on 48 hours post transfection.

FIG. 2(C) shows expression of nucleolin in S-oligos transfected NPC cells which is analyzed by Western blotting on 48 hours post transfection.

FIG. 3 demonstrates the inhibitory effect of nucleolin antisense transfection on nucleolin expression in NPC-cells.

FIG. 3(A) shows NPC cells transfected with 250 nM nucleolin antisense/sense S-oligos for 4 hours and incubated in culture medium containing 250 nM S-oligos for 1,2,3 and 4 days, respectively.

FIG. 3(B) shows effects of nucleolin S-oligos on proliferation on NPC-TW01 cells by MTT assay.

FIG. 4 shows that mice implanted with tumor cells treated with sense S-oligo, PBS and antisense S-oligos, respectively.

FIG. 4(A) shows change of implanted tumor size after treatment.

FIG. 4(B) shows tumor mass in mice of various treatment on day 38 after treatment.

### DETAILED DESCRIPTION OF THE PREFERRED EMBODIMENT

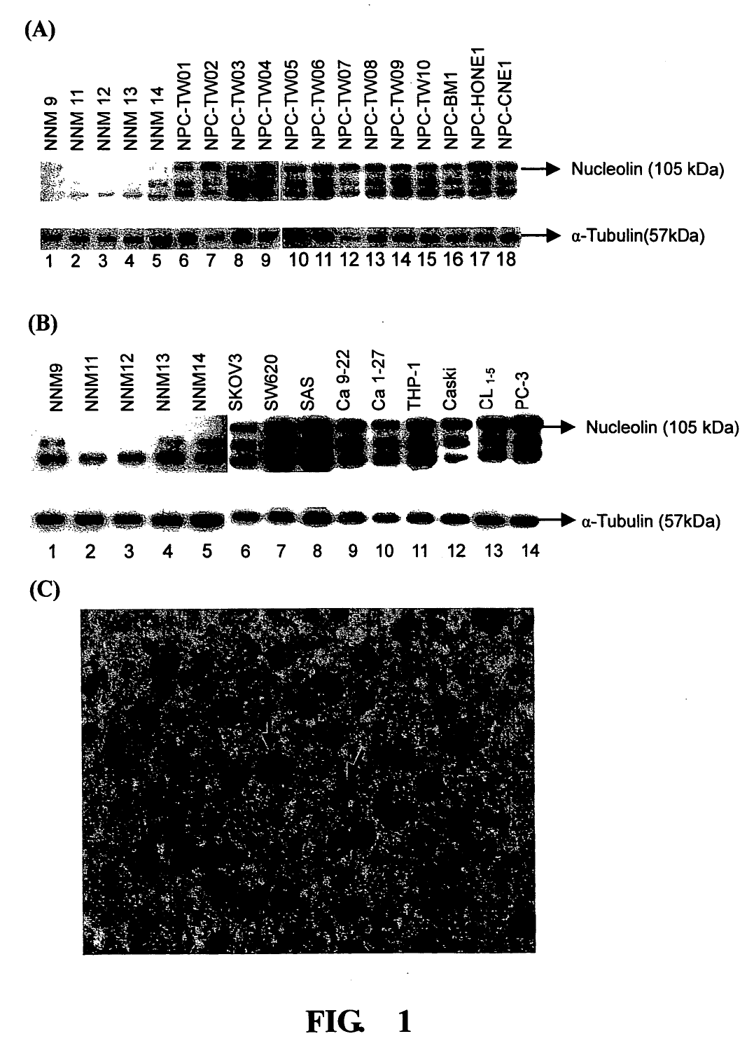

Referring to FIG. 1, Western blot analysis of the nucleolin expression in normal mucosal epithelial cells and NPC cells, and immunohistochemical localization of nucleolin in biopsy specimen are shown.

The results show in FIG. 1(A) that NPC cells (namely NPC-TW01-10, NPC-CGBM-1, NPC-HONE1 and NPC-CNE-1) have significant 105 kDa stained band and two degraded nucleolin bands (70 kDa and 50 kDa), while in the normal epithelial cells (namely NNM 9, 11, 12, 13 and 14, from National Taiwan University) one very weak 105 kDa band and two degraded bands are observed.

Seven non-NPC cancer cells are used to examine the expression of the nucleolin, including ovarian cancer cell line (SKOV3), colon cancer line (SW620), oral cancer line (SAS, Ca 9-22 and Ca 1-27), uterine cervical cancer line (CaSki), pulmonary adenocarcinoma line (CL-1-5), prostate cancer line (PC-3), and leukemia (THP-1). Western blot analysis of all these seven cancer cell lines shows strong intensity of stained protein bands similar to that 105 kDa protein band found in NPC cells, while in the normal epithelial control group, only two weak degraded bands are observed (FIG. 1(B), lane 1 to 5).

Besides the nucleolin expression analysis, the present invention also applies immunohistochemical localization to analyze the nucleolin expression in biopsy specimen. Similar results of immunohistochemical localization are observed, in which strong immunoreactivity of the anti-nucleolin reaction product appears in the nuclei and nucleoli of the cancer cells (arrowheads), while stromal cells are only weakly stained (FIG. 1(C)).

These results demonstrate that all the tested cancer cells have higher levels of nucleolin expression. Furthermore, the immunohistochemical localization of nucleolin in NPC biosepy specimens confirm the statement. All the phenomena strongly support that expression of nucleolin is closely related to the tumor cells proliferation.

As described above, level of nucleolin expression is in associated with the tumor cell proliferation. The present invention, therefore, designs an antisense fragment of nucleotide that is specifically complementary to the nucleolin mRNA, which would prohibit its translation, thus consequently reducing the growth rate of the tumor cells.

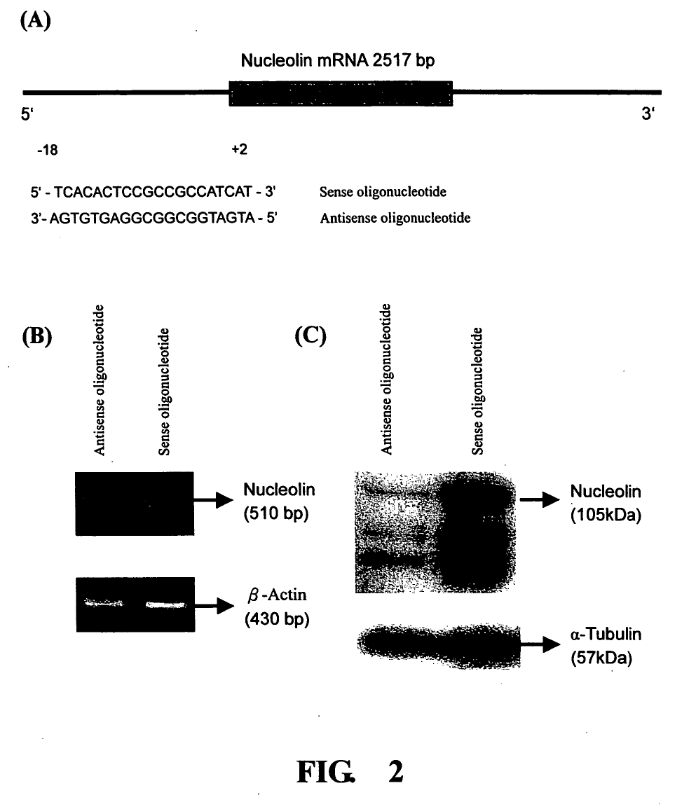

To demonstrate the concept described above, the nucleolin antisense S-oligos of the present invention is tested for its complementary binding to nucleolin mRNA and subsequent inhibition of corresponding protein translation. Another sense S-oligos is used as control. Both of the nucleolin antisense S-oligos and sense S-oligos are an oligonucleotide with 20 mers, which are designated as SEQ ID NO:1 and SEQ ID NO:2, respectively (FIG. 2(A)).

Transfection of the nucleolin antisense and sense S-oligos (250 nM) respectively is performed using Oligofectamine (Life Technologies) as the carrier. After transfection, levels of nucleolin RNA and protein expression are analyzed by RT-PCR and Western blotting. It is found that expression of the 510 bp nucleolin RNA band is significantly suppressed in the nucleolin antisense S-oligos treated cells (FIG. 2(B), Lane 1), whereas the same 510 bp band is revealed in the control experiment (FIG. 2(B), Lane 2). Similarly, in the Western blot analysis, the nucleolin antisense S-oligos treated cells show a weak 105 kDa band and two weak degraded bands of nucleolin (FIG. 2(C), Lane1), while the sense S-oligos treated cells show a strong 105 kDa and two clear degraded bands of nucleolin(FIG. 2(C), Lane 2).

Based on the results of antisense S-oligos transfection experiment, it is confirmed that nucleolin antisense S-oligos of the present invention indeed hybridizes to nucleolin mRNA and effectively suppresses its protein synthesis consequently, and the idea can be applied in other tumor cells as well. Furthermore, the inhibition of tumor cell proliferation can be easily achieved by using tiny amount of the nucleolin antisense S-oligos (250 nM).

To confirm the effectiveness of the nucleolin antisense S-oligos (SEQ ID No:1) provided by the present invention, subsequently, it is important to further examine that the nucleolin antisense S-oligos can survive and keep its bioactivity in vitro and inhibit the proliferation of the tumor cells.

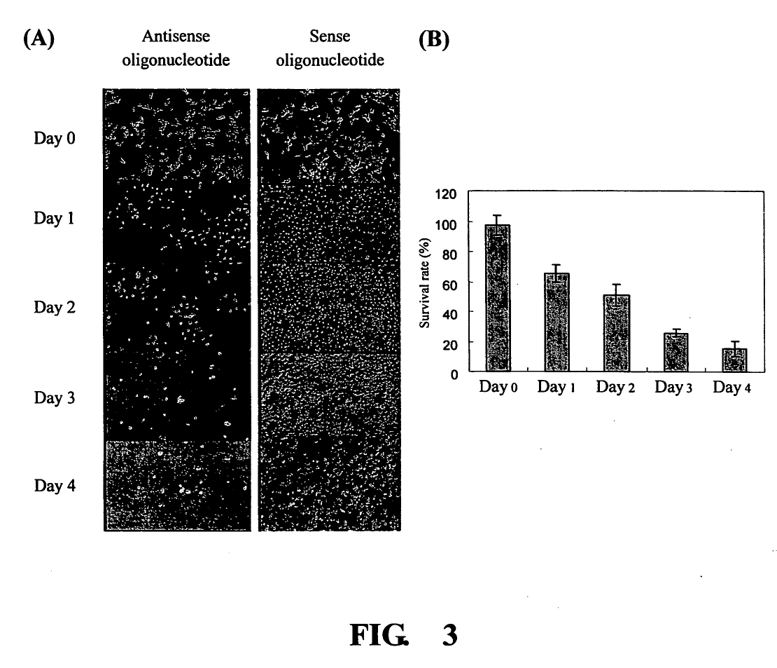

FIG. 3 shows the effect of the nucleolin antisense S-oligos on the growth of the NPC cells. Nucleolin sense/antisense S-oligos (250 nM) respectively is transfected to the NPC cells, and morphological change and cell number are monitored in the following 1-4 days after transfection. It is observed that the antisense-treated cell numbers are remarkably reduced daily with clear apoptotic change, while the sense-treated cells show normal cell proliferation (FIG. 3(A)).

Survival rate of the nucleolin sense/antisense treated NPC-TW01 and NNM cells are further analyzed using MTT assay [3-(4,5-dimethylthiazol-2-yl)-2,5-diphenyl-tetrazolium bromide, MTT]. The results show that nucleolin antisense treatment suppresses growth of tumor cells and is time dependent (FIG. 3(B)), while the NNM cells show no or only little suppression of cell growth.

The experiment described above indicates that the nucleolin antisense S-oligos (SEQ ID No:1) provided by the present invention is effective to suppress tumor cell growth in vitro. Therefore, inhibition of tumor cell growth by antisense S-oligos (SEQ ID No:1) of the present invention is further tested in vivo. SCID mice are implanted with NPC-TW01 tumor cells. When the tumor lumps are well developed, the nucleolin sense/antisense S-oligos are injected into the mice separately by intra-venous injection (i.v.), and then size and weight of the tumor lumps are monitored.

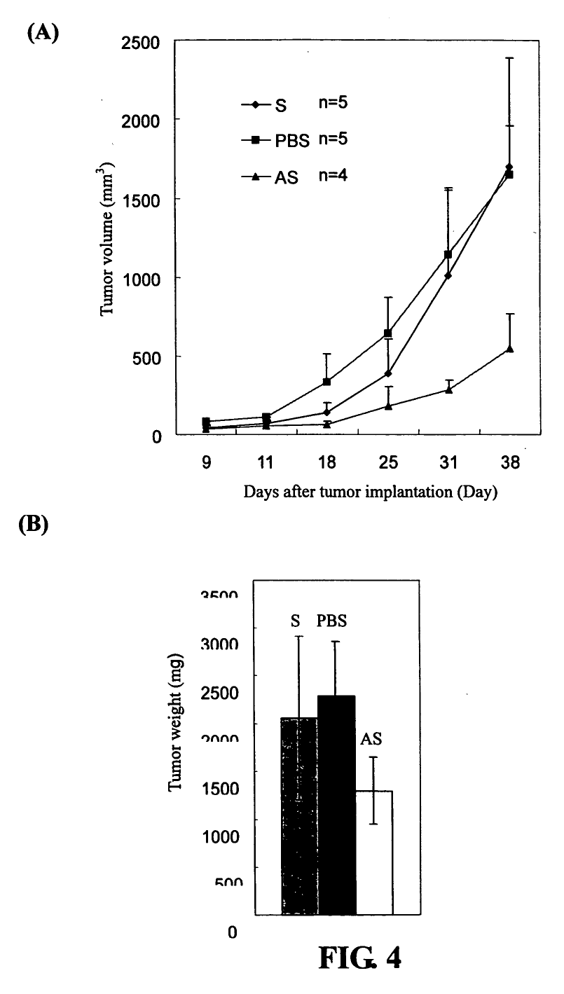

As shown in FIG. 4(A), growth of tumor lump in mice treated with the nucleolin antisense S-oligos is inhibited, while the mice treated with sense S-oligos or phosphate buffer solution are not affected.

The tumor-implanted SCID mice are sacrificed on day 38 after implantation, and the tumor masses are measured. The results (FIG. 4(B)) show that the tumor size in the nucleolin antisense treated group is significantly smaller than that of the group injected with sense S-oligos or phosphate buffer solution.

### EXAMPLE 1

Nucleolin Antisense Oligonucleotide on Suppression of Nucleolin Expression

Based on the sequence of nucleolin mRNA, the inventor designs a specific nucleolin antisense sequence (SEQ ID NO:1) and synthesizes the sequence using Sigma Genosys. Nucleolin sense oligonucleotide (SEQ ID NO:2) is also synthesized for control experiment.

Treansfection of the sense/antisense S-oligos (250 nM) is carried out by using transfection reagent Oligofectamine as a vector. Before transfection, NPC-TW01 cells and NNM cells are cultivated in 6-well plates at a density of 5×10⁴ cells for 24 hours. The transfection mixture is prepared in Opti-MEM containing 4 μl Oligofectamine per 250 nM oligonucleotide according to the manufacturer's instructions. Transfection is allowed to carry out for 4 hours after the addition of the transfection mixture in a final volume of 1 ml per well. Then, 10% fetal calf serum medium is added without removing the transfection mixture. Nucleolin RNA and the corresponding protein synthesis at 48 hours after transfection are analyzed by RT-PCR and Western blotting.

The results indicate that the nucleolin mRNA is suppressed in the antisense S-oligos treated cells (FIG. 2(B), Lane 1), whereas a clear 510 bp band of nucleolin is observed in sense S-oligos treated cells (FIG. 2(B), Lane 2). Similar results are observed in Western blotting, wherein the antisense treated cells appear a weak 105 kDa band and two weak degraded bands of nucleolin (FIG. 2(C), Lane 1), while the sense treated cells reveal a strong 105 kDa and two clear degraded bands of nucleolin (FIG. 2(C), Lane 2).

### EXAMPLE 2

Analysis of Growth Inhibitory Effect of Nucleolin Antisense on Tumor Cells by MTT Assay

Tumor cells at density of 4×10³ cells are incubated in 96-well microplates and allowed to grow overnight. Tumor cells are then treated with nucleolin antisense S-oligos for 12, 24, and 48 hours. After treatment, 100 μl of 2 mg/ml MTT in DMEM containing 5% of FBS serum is added to each well, followed by incubation for 2.5 hours at 37° C. The formazan crystals then dissolved in DMSO. The absorbance is determined with a reader at 540 nm. Absorbance values are normalized to the values obtained for the sense oligonucleotides-treated cells to determine the percentage of survival. Each assay is performed in triplicate.

The results show that after nucleolin antisense treatment, tumor cell growth is suppressed and its effectiveness is time dependent (FIG. 3(B)), while the NNM cells show no or only little suppression of cell growth.

### EXAMPLE 3

The Inhibitory Effect of Antisense S-oligos on Tumor Cell Proliferation in vivo

SCID mice (female, 4-6 weeks old) are purchased from National Taiwan University Hospital Laboratory Animal Center and accommodated for 7 days for environmental adjustment before study. Cultured NPC TW-01 cells are trypsinized, resuspended in serum-free DMEM and then injected s.c. (subcutaneous injection) (1×10⁷ cells, total volume 0.2 ml) into the mice.

When the implanted NPC-TW01 tumor cells in the mice grow into lump of a size approximately 80-100 mm³, mice are weighed and treated with the nucleolin sense/antisense S-oligos or phosphate buffer solution, respectively, by i.v. injection (10 mg/kg weight) every two days. Growth of tumor size and body weight of the mice are observed for 35 days.

FIG. 4(A) shows that tumor volume of the nucleolin antisense S-oligos treated mice is suppressed, meanwhile no inhibitory effect of tumor growth is detected in sense S-oligos or phosphate buffer treated mice.

When these treated mice are sacrificed on day 38 and the tumor mass is weighed, it is found that tumor mass of the nucleolin antisense S-oligos treated mice is smaller than the ones treated with sense S-oligos or phosphate buffer (FIG. 4(B)).

1. A nucleolin antisense oligonucleotide for proliferation inhibition of tumor cells comprising at least one nucleotide sequence as designated SEQ ID NO:1, wherein the nucleotide sequence can hybridize to nucleolin mRNA specifically and suppress nucleolin expression.

2. The nucleolin antisense oligonucleotide of claim 1, wherein the nucleolin antisense oligonucleotide is effective in inhibition of tumor cell proliferation.

3. The nucleolin antisense oligonucleotide of claim 2, wherein the nucleolin antisense is effective in reduction of tumor size in vivo.

4. The nucleolin antisense oligonucleotide of claim 1, wherein the specific nucleolin mRNA sequence comprises translation start codon of the mRNA.

5. The nucleolin antisense oligonucleotide of claim 1, wherein the nucleolin antisense is a phosphorothioate-modified oligonucleotide.

6. The nucleolin antisense oligonucleotide of claim 1, wherein the tumor cells are nasopharyngeal carcinoma cells, ovarian cancer cells, colon cancer cells, oral cancer cells, uterine cervical cancer cells, pulmonary adenocarcinoma cells, prostate cancer cells or leukemia cells.

7. A vector of nucleolin antisense oligonucleotide for tumor growth inhibition, wherein the nucleolin antisense oligonucleotide is SEQ ID NO:1.

8. A therapeutic composition comprising the nucleolin antisense oligonucleotide of claim 1 and a pharmaceutically acceptable carrier or diluent.

9. A method for proliferation inhibition of tumor cells, wherein the method comprising the steps of: hybridizing nucleolin antisense oligonucleotides of claim 1 to nucleolin mRNAs of the tumor cells, inhibiting expression of the nucleolin, and inhibiting proliferation of tumor cells.

10. The method of claim 9, wherein the tumor cells are nasopharyngeal carcinoma cells, ovarian cancer cells, colon cancer cells, oral cancer cells, uterine cervical cancer cells, pulmonary adenocarcinoma cells, prostate cancer cells or leukemia cells.


SEQUENCE LISTING (2 sequences)

SEQ ID NO: 1 - DNA, 20 bp, Artificial Sequence
  Antisense fragment which sequence is specifically complementary to the nucleolin mRNA
atgatggcggcggagtgtga

SEQ ID NO: 2 - DNA, 20 bp, Artificial Sequence
  Sense fragment which sequence is similar to the nucleolin mRNA
tcacactccgccgccatcat